(19) United States
(12) Patent Application Publication
(10) Pub. No.: US 2011/0263903 A1
(43) Pub. Date: Oct. 27, 2011

(54) PROCESS FOR PREPARING 4,4'-DICHLORODIPHENYL SULFONE

(75) Inventors: Patrick Deck, Sao Paulo City (BR); Heiner Schelling, Kirchheim (DE); Florian Garlichs, Neustadt (DE)

(73) Assignee: BASF SE, Ludwigshafen (DE)

(21) Appl. No.: 13/091,694

(22) Filed: Apr. 21, 2011

Related U.S. Application Data
(60) Provisional application No. 61/326,686, filed on Apr. 22, 2010.

(51) Int. Cl. C07C 315/00

(52) U.S. Cl. 568/35

(57) ABSTRACT

The invention relates to a process for preparing 4,4′-dichlorodiphenyl sulfone proceeding from monochlorobenzene, wherein the content in the monochlorobenzene used of hydrocarbons having from 5 to 8 carbon atoms is at most 100 ppm; based on the total weight of the monochlorobenzene used, including the secondary components.

The present invention further relates to the use of monochlorobenzene with the properties mentioned for preparation of 4,4′-dichlorodiphenyl sulfone.

The invention relates to a process for preparing 4,4′-dichlorodiphenyl sulfone proceeding from monochlorobenzene, wherein the content in the monochlorobenzene used of hydrocarbons having from 5 to 8 carbon atoms is at most 100 ppm, based on the total weight of the monochlorobenzene used, including the secondary components.

The present invention further relates to the use of monochlorobenzene with the properties mentioned for preparation of 4,4′-dichlorodiphenyl sulfone.

4,4′-Dichlorodiphenyl sulfone is used especially as a monomer in the synthesis of polyarylene ether sulfones. Examples of commercial significance are polyether sulfone (polymerization of 4,4′-dihydroxydiphenyl sulfone with 4,4′-dichlorodiphenyl sulfone), polysulfone (polymerization of bisphenol A with 4,4′-dichlorodiphenyl sulfone) and polyphenylene sulfone (polymerization of 4,4′-dihydroxybiphenyl with 4,4′-dichlorodiphenyl sulfone). 4,4′-Dichlorodiphenyl sulfone is thus a central element for the preparation of these industrial polymers.

The preferred reactant for the preparation of polyarylene ether sulfones is high-purity 4,4′-dichlorodiphenyl sulfone, firstly since the 4,4′ isomer forms exclusively linear, nonangular polymers which have the desired product properties, for example chemical and thermal stability, high dimensional stability and low flammability, and secondly since impurities frequently lead to undesired discoloration and to a deterioration in the properties of the polymers.

Processes for preparing 4,4′-dichlorodiphenyl sulfone proceeding from monochlorobenzene are known from the prior art. The known processes comprise, more particularly, the preparation proceeding from monochlorobenzene and a sulfonating agent via 4-chlorobenzenesulfonic acid as an intermediate which is generally not isolated.

U.S. Pat. No. 2,593,001 describes a continuous process for preparing diaryl sulfones by reaction of aromatic sulfonic acids with aromatics, wherein the water of reaction is removed continuously from the reaction zone by the aromatic compound added in gaseous form in countercurrent.

U.S. Pat. No. 2,971,985 discloses the synthesis of dichlorodiphenyl sulfone using SO₃, dimethyl sulfate and monochlorobenzene. EP 0 381 049 A1 likewise describes a process for preparing 4,4-dichlorodiphenyl sulfone. This involves reacting sulfur trioxide, dimethyl sulfate and chlorobenzene at 50 to 100° C. Reaction outputs from the synthesis of 4,4′-dichlorodiphenyl sulfone according to U.S. 2,593,001, U.S. Pat. No. 2,971,985 or EP 0 381 049 A1 are typically colored. This color resulting from highly coloring by-products in very low concentrations can be prevented by means of workup by crystallization only incompletely or only in a very complex manner.

In order to obtain 4,4′-dichlorodiphenyl sulfone in a quality needed for use as a monomer unit, a workup of the crude product initially obtained, i.e. a mixture comprising 4,4′-dichlorodiphenyl sulfone, is always required. For this purpose, the prior art discloses different processes.

U.S. Pat. No. 4,937,387 describes, building on the synthesis according to U.S. Pat. No. 2,593,001, the separation of the reaction mixture by addition of water, separation of the two liquid phases formed and subsequent isolation of dichlorodiphenyl sulfone.

Mixtures of the dichlorodiphenyl sulfone isomers can be worked up, for example, by crystallization with or from alcohols, such that increased purities of the desired 4,4′-dichlorodiphenyl sulfone are obtained. EP-A 279387 describes this type of purification by recrystallization. U.S. Pat. No. 4,016,210 describes the crystallization of 4,4′-dichlorodiphenyl sulfone from a reaction mixture which results from the reaction of chlorobenzenesulfonic acid and chlorobenzene.

A disadvantage of the known processes for preparing 4,4′-dichlorodiphenyl sulfone is thus the complexity of the workup which follows the production of the crude product initially obtained. The degree of complexity is determined to a high degree by the type and amount of secondary components. Secondary components of 4,4′-dichlorodiphenyl sulfone can typically be removed by crystallization only with difficulty. An additional complicating factor is that coloring secondary components even in very small amounts discolor the 4,4′-dichlorodiphenyl sulfone and any polymers prepared therefrom in an undesired manner. It would thus be desirable to provide a process for preparing 4,4′-dichlorodiphenyl sulfone, which reduces or avoids the formation of coloring secondary components of 4,4′-dichlorodiphenyl sulfone.

It was thus an object of the present invention to provide a process for preparing 4,4′-dichlorodiphenyl sulfone, which has the aforementioned disadvantages to a lesser degree, if at all.

More particularly, the process should provide 4,4′-dichlorodiphenyl sulfone in high purity in a simple manner in process technology terms. The proportion of secondary components of 4,4′-dichlorodiphenyl sulfone formed in the preparation, especially coloring secondary components, should be reduced or even avoided compared to the prior art.

The aforementioned objects are achieved by the process according to the invention for preparing 4,4′-dichlorodiphenyl sulfone. Preferred embodiments can be inferred from the claims and the description which follows. Combinations of preferred embodiments do not leave the scope of the present invention.

The process according to the invention for preparing 4,4′-dichlorodiphenyl sulfone proceeds from monochlorobenzene, wherein the content in the monochlorobenzene used of hydrocarbons having from 5 to 8 carbon atoms is at most 100 ppm, based in each case on the total weight of the monochlorobenzene used, including the secondary components.

In the context of the present invention, the content in the monochlorobenzene used of hydrocarbons having from 5 to 8 carbon atoms is always calculated as the weight of the hydrocarbons (in the ppm unit, which in the context of the present invention denotes parts by weight) and based on the total weight of the monochlorobenzene used, including all secondary components.

In the context of the present invention, the content in the monochlorobenzene used of hydrocarbons is determined by means of gas chromatography separation and subsequent detection by means of flame ionization. The secondary components are determined quantitatively by means of the addition of a standard. Corresponding quantitative methods are known to those skilled in the art. The detection limit typically achievable in this method is 0.1 ppm.

In principle, useful processes in the context of the process according to the invention are all known processes for preparing 4,4′-dichlorodiphenyl sulfone which proceed from monochlorobenzene, especially processes which comprise the reaction of monochlorobenzene with a sulfonating agent, especially those which proceed via 4-chlorobenzenesulfonic acid as an intermediate. Corresponding processes are known to those skilled in the art. Processes used with preference for preparing 4,4′-dichlorodiphenyl sulfone are described below in the context of step (b) of a preferred embodiment.

The monochlorobenzene used preferably at the same time has a purity of at least 99.8% by weight. In the context of the present invention, a purity of at least 99.8% by weight means that the monochlorobenzene used consists of the chemical compound monochlorobenzene at least to an extent of 99.8% by weight. The percentage by weight is based on the total weight of the monochlorobenzene used. It is known to those skilled in the art that the monochlorobenzene used, as well as the chemical compound monochlorobenzene, also comprises impurities, referred to hereinafter as secondary components. The purity figure is thus always based on the total weight of the monochlorobenzene used and comprises all secondary components including the hydrocarbons mentioned.

According to the present invention, the proportion of secondary components in the monochlorobenzene used in the preparation of 4,4′-dichlorodiphenyl sulfone is preferably at most 0.2% by weight, based on the total weight of the monochlorobenzene used, including all secondary components. The proportion of hydrocarbons, which by definition consist exclusively of carbon and hydrogen, having 5 to 8 carbon atoms in the total weight of the monochlorobenzene used is, in accordance with the invention, at most 100 ppm.

The proportion of hydrocarbons having 5 to 8 carbon atoms in the total weight of the monochlorobenzene used is preferably at most 80 ppm in total, more preferably at most 50 ppm, especially at most 20 ppm, most preferably at most 10 ppm, especially at most 5 ppm, based in each case on the total weight of the monochlorobenzene used, including the secondary components.

In the context of the present invention, the lower limit for the inventive hydrocarbons having 5 to 8 carbon atoms is in principle zero. However, it is possible only with a very high level of complexity, if at all, to attain the lower limit of zero. In this respect, a customary lower limit resulting from process technology considerations for the proportion of hydrocarbons having 5 to 8 carbon atoms in the total weight of the monochlorobenzene used is, for example, 0.1 ppm or 0.01 ppm, based on the total weight of the monochlorobenzene used, including the secondary components. To determine the content of secondary components present at less than 0.1 ppm, the person skilled in the art turns to known trace analysis methods.

In the context of the present invention, hydrocarbons are understood to mean compounds formed exclusively from carbon and hydrogen. When types of atoms other than carbon and hydrogen are present, the corresponding compounds are named differently, for example halogenated hydrocarbons.

Hydrocarbons having 5 to 8 carbon atoms comprise especially saturated linear aliphatic hydrocarbons (n-pentane, n-hexane, n-heptane, n-octane), saturated branched aliphatic hydrocarbons, saturated cycloaliphatic hydrocarbons, unsaturated hydrocarbons which derive from the aforementioned saturated hydrocarbons by theoretical elimination of hydrogen, and aromatic hydrocarbons.

The group of the hydrocarbons having 5 to 8 carbon atoms comprises especially saturated or unsaturated cycloaliphatic hydrocarbons and saturated or unsaturated, linear or branched aliphatic hydrocarbons, hereinafter referred to collectively as (cyclo)aliphatic hydrocarbons, the (cyclo)aliphatic hydrocarbons having 5 to 8 carbon atoms being present preferably to an extent of at most 80 ppm in total, more preferably at most 50 ppm, especially at most 20 ppm, most preferably at most 10 ppm, based in each case on the total weight of the monochlorobenzene used, including the secondary components.

It is additionally preferred to restrict the proportion of hydrocarbons having 6 or 7 carbon atoms in the monochlorobenzene used to a total of at most 80 ppm, preferably at most 50 ppm, especially at most 20 ppm, even more preferably at most 10 ppm, especially at most 5 ppm.

The monochlorobenzene used preferably has a purity of at least 99.9% by weight, especially 99.95% by weight, based on the total weight of the monochlorobenzene used, including the secondary components. The monochlorobenzene used more preferably has a purity of at least 99.99% by weight, especially at least 99.995% by weight, most preferably at least 99.999% by weight, based on the total weight of the monochlorobenzene used, including the secondary components. In the context of the present invention, the proportion of the secondary components in the monochlorobenzene used is always determined by means of gas chromatography separation and subsequent detection by means of flame ionization. The secondary components are determined quantitatively by means of the addition of a standard.

Hydrocarbons having 5 to 8 carbon atoms have a particularly unfavorable influence on the color number of the 4,4′-dichlorodiphenyl sulfone formed in the process according to the invention.

Particularly relevant hydrocarbons with regard to influence on the color number have been found to be saturated cycloaliphatic hydrocarbons of the empirical formula C₆H₁₂ and C₇H₁₄, unsaturated hydrocarbons of the empirical formula C₇H₁₂ and saturated aliphatic, linear or branched hydrocarbons of the empirical formulae C₅H₁₂ and C₆H₁₄. The content in the monochlorobenzene used of the (cyclo)aliphatic compounds having 6 or 7 carbon atoms mentioned in the preceding sentence is preferably at most 80 ppm in total, more preferably at most 50 ppm, especially at most 20 ppm, even more preferably at most 10 ppm, especially at most 5 ppm, based in each case on the total weight of the monochlorobenzene used, including the secondary components.

The (cyclo)aliphatic compounds having 6 carbon atoms, especially cyclohexane, methylcyclopentane, methylcyclopentene, linear or branched hexane, and the (cyclo)aliphatic compounds having 7 carbon atoms, especially methylcyclohexane, methylcyclohexene and linear or branched heptane, have a particularly unfavorable influence on the color number of the 4,4′-dichlorodiphenyl sulfone formed.

In the context of the present invention, in addition, the proportion of halogenated hydrocarbons having 5 to 8 carbon atoms apart from monochlorobenzene in the monochlorobenzene used is preferably at most 100 ppm, more preferably at most 50 ppm, especially at most 20 ppm, even more preferably at most 10 ppm, especially at most 5 ppm, based in each case on the total weight of the monochlorobenzene used, including the secondary components. At the same time, the monochlorobenzene used has a proportion of hydrocarbons having 5 to 8 carbon atoms within the preferred range explained above.

Halogenated hydrocarbons having 5 to 8 carbon atoms apart from monochlorobenzene are compounds comprising carbon, hydrogen and at least one halogen atom, especially chlorine and/or bromine, especially chlorine. This compound class comprises especially monobromobenzene and dichlorobenzene.

The present invention further provides a process for preparing 4,4′-dichlorodiphenyl sulfone, comprising at least the following steps:

(a) purifying monochlorobenzene to obtain monochlorobenzene with the inventive properties in relation to the secondary components, and then

(b) converting the monochlorobenzene obtained in step (a) to obtain a mixture comprising 4,4′-dichlorobenzenesulfone, and then

(c) preferably removing 4,4′-dichlorobenzenesulfone from the mixture obtained in step (b).

The individual steps are explained hereinafter.

Step (a)

Methods for purifying monochlorobenzene are known per se to those skilled in the art. It is essential to the invention that the monochlorobenzene used in step (b) has the abovementioned inventive or preferred properties in relation to purity and secondary components.

The monochlorobenzene is preferably purified by distillation in step (a). Corresponding distillative processes are likewise known to those skilled in the art. Useful processes are especially a batch distillation or distillative purification by means of a dividing wall column. Distillative purification by means of a dividing wall column is particularly preferred since this technique can remove both the high boiler fraction and the low boiler fraction in only one step.

Step (b)

Processes for preparing 4,4′-dichlorodiphenyl sulfone proceeding from monochlorobenzene are known per se and can be implemented as process steps (b) of the present process.

The step (b) mentioned relates to the conversion of the monochlorobenzene obtained in step (a) to a mixture comprising 4,4′-dichlorodiphenyl sulfone (crude product). The by-products formed in the conversion proceeding from monochlorobenzene are especially 2,4′-dichlorodiphenyl sulfone and/or 3,4′-dichlorodiphenyl sulfone (incorrect isomers of 4,4′-dichlorodiphenyl sulfone). In addition, 2-chlorobenzenesulfonic acid, 3-chlorobenzenesulfonic acid and/or 4-chlorobenzenesulfonic acid are generally formed. The abovementioned hydrocarbons having 5 to 8 carbon atoms, as secondary components of monochlorobenzene, additionally lead to a complex spectrum of coloring compounds in the crude product mentioned.

The reaction of monochlorobenzene with a sulfonating agent typically forms 4-chlorobenzenesulfonic acid. However, this unavoidably forms incorrect isomers of 4-monochlorobenzenesulfonic acid as by-products which are undesired per se. Subsequently, the 4-chlorobenzenesulfonic acid and the 2-chlorobenzenesulfonic acid and 3-chlorobenzenesulfonic acid isomers thereof are reacted with monochlorobenzene to give 4,4′-dichlorodiphenyl sulfone, which forms the abovementioned incorrect isomers of 4,4′-dichlorodiphenyl sulfone. Monochlorobenzenesulfonic acid can also form as an intermediate which is not isolated.

In a preferred first embodiment, 4,4′-dichlorodiphenyl sulfone is prepared by reacting 4-chlorobenzenesulfonic acid with monochlorobenzene. The reaction is preferably performed in a countercurrent column, in which case the water of reaction continuously is stripped out overhead by the aromatic added in gaseous form in the bottom of the column. For the synthesis of 4,4′-dichlorodiphenyl sulfone, 4-chlorobenzenesulfonic acid or else sulfuric acid can be added at the top of the column. The latter reacts in the column with monochlorobenzene first to give monochlorobenzenesulfonic acid, which subsequently likewise reacts with monochlorobenzene to give dichlorodiphenyl sulfone. The corresponding process is described, for example, in U.S. Pat. No. 2,593,001, the content of which is hereby fully incorporated.

In a second preferred embodiment, dichlorodiphenyl sulfone is prepared using SO₃, dimethyl sulfate and monochlorobenzene. Preferably, SO₃ and dimethyl sulfate are first allowed to react under moderate conditions in a molar ratio of 2 to 1. In the course of this, some of the SO₃ reacts with dimethyl sulfate to form the corresponding pyrosulfate. The rest of the SO₃ remains dissolved in the liquid which forms. This mixture is subsequently added at temperatures below 100° C. to 2 mol of monochlorobenzene per 2 mol of SO₃ and 1 mol of dimethyl sulfate. The dissolved SO₃, the dimethyl pyrosulfate and the monochlorobenzene form 1 mol of dichlorodiphenyl sulfone and 2 mol of monomethyl sulfate. The reaction mixture is subsequently passed into water. Dichlorodiphenyl sulfone precipitates out. It is filtered off and dried. The corresponding process is described, for example, in U.S. Pat. No. 2,971,985, the content of which is hereby fully incorporated.

The person skilled in the art is aware that the reactions explained above can be combined with other workup processes than those detailed above.

Step (c)

In the course of step (c), 4,4′-dichlorodiphenyl sulfone is preferably removed from the mixture obtained in step (b), i.e. the crude product which comprises the desired reaction product and by-products is worked up.

Suitable processes for workup of the crude product are known per se to those skilled in the art.

In one embodiment, the reaction mixture is separated by adding water and separating the two liquid phases which form. The aqueous phase comprises unconverted monochlorobenzenesulfonic acid. The water is evaporated off and the monochlorobenzenesulfonic acid is recovered as a feedstock. Dichlorodiphenyl sulfone can be isolated from the organic phase, which consists predominantly of monochlorobenzene and dichlorodiphenyl sulfone. A corresponding process is described, for example, in U.S. Pat. No. 4,937,387, the content of which is hereby fully incorporated.

4,4′-Dichlorodiphenyl sulfone can be removed from the crude product, for example, by chromatography. The removal is preferably effected by recrystallization, as described, for example, in EP 279 387, the content of which is hereby fully incorporated.

### EXAMPLES

### Example 1

The monochlorobenzene used in example 1 comprised, according to gas chromatography separation and subsequent detection by means of flame ionization, the following secondary components: 30 ppm of methylcyclohexane and 10 ppm of methylcyclohexene.

126.1 g (1 mol) of dimethyl sulfate were heated to 70-75° C. with exclusion of air humidity and 80.1 g (1 mol) of liquid sulfur trioxide were added at this temperature. The mixture was left to stir at this temperature for 30 min and then cooled to 20° C. A further 80.1 g (1 mol) of liquid sulfur trioxide were added at such a rate that the temperature does not exceed 30° C. The reaction mixture was added within 20 min to 225.1 g (2 mol) of monochlorobenzene preheated to 50° C. Subsequently, the mixture was stirred at 50° C. for another 1 h. After cooling, a yellowish-orange solution was obtained.

### Example 2

The monochlorobenzene used in example 2 comprised, according to gas chromatography separation and subsequent detection by means of flame ionization, the following secondary components: hydrocarbons having from 5 to 8 carbon atoms <1 ppm in total.

126.1 g (1 mol) of dimethyl sulfate were heated to 70-75° C. with exclusion of air humidity and 80.1 g (1 mol) of liquid sulfur trioxide were added at this temperature. The mixture was left to stir at this temperature for 30 min and then cooled to 20° C. A further 80.1 g (1 mol) of liquid sulfur trioxide were added at such a rate that the temperature does not exceed 30° C. The reaction mixture was added within 20 min to 225.1 g (2 mol) of monochlorobenzene preheated to 50° C. Subsequently, the mixture was stirred at 50° C. for another 1 h. After cooling, a pale yellow solution was obtained.

### Example 3 (Comparative Example)

126.1 g (1 mol) of dimethyl sulfate were heated to 70-75° C. with exclusion of air humidity and 80.1 g (1 mol) of liquid sulfur trioxide were added at this temperature. The mixture was left to stir at this temperature for 30 min and then cooled to 20° C. A further 80.1 g (1 mol) of liquid sulfur trioxide were added at such a rate that the temperature does not exceed 30° C. The reaction mixture was added within 20 min to a mixture of 1.68 g (0.02 mol) of cyclohexane and 225.1 g (2 mol) of monochlorobenzene preheated to 50° C. (identical to example 1). Subsequently, the mixture was stirred at 50° C. for another 1 h. After cooling, a dark orange solution was obtained.

The process according to the invention affords a crude product of 4,4′-dichlorodiphenyl sulfone which has a much lower color number and hence a much higher purity. The crude product of 4,4′-dichlorodiphenyl sulfone obtained in accordance with the invention can be subjected to known processes for workup and isolation of pure 4,4′-dichlorodiphenyl sulfone. The workup can be effected with lower complexity, especially by virtue of the known processes being performed more rapidly and/or in a smaller number of repeats.

### Example 4

The monochlorobenzene used in example 4 comprised, according to gas chromatography separation and subsequent detection by means of flame ionization, the following secondary components: 20 ppm of hexane, 20 ppm of cyclohexane, 10 ppm of methylcyclohexane, 10 ppm of bromobenzene and 10 ppm of dichlorobenzene.

With exclusion of air humidity, 680 g (8.5 mol) of freshly distilled sulfur trioxide were added at a maximum of 45° C. to 1914 g (17 mol) of monochlorobenzene. The resulting dark gray/brown solution (chlorobenzenesulfonic acid in monochlorobenzene) can be used to synthesize dichlorodiphenyl sulfone, for example according to U.S. Pat. No. 2,593,001.

### Example 5

The monochlorobenzene used in example 5 was obtained by additionally subsequently distilling the monochlorobenzene used in example 1 over P₂O₅ in a batch distillation column and hence purifying it. The monochlorobenzene used in example 5 comprised, according to gas chromatography separation and subsequent detection by means of flame ionization, the following secondary components: hydrocarbons having from 5 to 8 carbon atoms <1 ppm.

With exclusion of air humidity, 680 g (8.5 mol) of freshly distilled sulfur trioxide were added at a maximum of 45° C. to 1914 g (17 mol) of monochlorobenzene. The pale brown solution (chlorobenzenesulfonic acid in monochlorobenzene) can be used to synthesize dichlorodiphenyl sulfone, for example according to U.S. Pat. No. 2,593,001.

### Example 6 (Comparative Example)

With exclusion of air humidity, 680 g (8.5 mol) of freshly distilled sulfur trioxide were added at a maximum of 45° C. to a mixture of 1914 g (17 mol) of monochlorobenzene (identical to example 5) and 8.4 g (0.1 mol) of cyclohexane. The resulting solution (chlorobenzenesulfonic acid in monochlorobenzene) was black-brown.

1.-13. (canceled)

14. A process for preparing 4,4′-dichlorodiphenyl sulfone which comprises proceeding from monochlorobenzene, wherein the content in the monochlorobenzene used of hydrocarbons having from 5 to 8 carbon atoms is at most 100 ppm, based on the total weight of the monochlorobenzene used, including the secondary components, to 4,4′-dichlorodiphenyl sulfone.

15. The process according to claim 14, wherein the content in the monochlorobenzene used of halogenated hydrocarbons having 5 to 8 carbon atoms apart from monochlorobenzene is at most 50 ppm and the content in the monochlorobenzene used of hydrocarbons having from 5 to 8 carbon atoms is at most 50 ppm, based on the total weight of the monochlorobenzene used, including the secondary components.

16. The process according to claim 14, wherein the content in the monochlorobenzene used of hydrocarbons having from 5 to 8 carbon atoms is at most 20 ppm, based on the total weight of the monochlorobenzene used, including the secondary components.

17. The process according to claim 14, wherein the content in the monochlorobenzene used of hydrocarbons having from 5 to 8 carbon atoms is at most 10 ppm, based on the total weight of the monochlorobenzene used, including the secondary components.

18. The process according to claim 14, wherein the monochlorobenzene used has a purity of at least 99.9% by weight, based on the total weight of the monochlorobenzene used, including the secondary components.

19. The process according to claim 17, wherein the monochlorobenzene used has a purity of at least 99.9% by weight, based on the total weight of the monochlorobenzene used, including the secondary components.

20. The process according to claim 14, wherein the monochlorobenzene used has a purity of at least 99.99% by weight.

21. The process according to claim 14, wherein the content in the monochlorobenzene used of (cyclo)aliphatic hydrocarbons having 6 or 7 carbon atoms is at most 50 ppm in total, based on the total weight of the monochlorobenzene used, including the secondary components.

22. The process according to claim 14, wherein the content in the monochlorobenzene used of (cyclo)aliphatic hydrocarbons having 6 or 7 carbon atoms is at most 10 ppm in total, based on the total weight of the monochlorobenzene used, including the secondary components.

23. A process for preparing 4,4′-dichlorodiphenyl sulfone comprising
(a) purifying monochlorobenzene to obtain monochlorobenzene wherein the content in the monochlorobenzene used of hydrocarbons having from 5 to 8 carbon atoms is at most 100 ppm, based on the total weight of the monochlorobenzene used, including the secondary components and then
(b) converting the monochlorobenzene obtained in step (a) to a mixture comprising 4,4′-dichlorodiphenyl sulfone.

24. The process according to claim 23, wherein step (b) is followed, in step (c), by the removal of 4,4′-dichlorodiphenyl sulfone from the mixture obtained in step (b).

25. The process according to claim 23, wherein the monochlorobenzene is purified by distillation in step (a).

26. The process according to claim 23, wherein the monochlorobenzene is purified by distillation in a dividing wall column in step (a).

27. A process for preparing 4,4′-dichlorodiphenyl sulfone which comprises reacting 4-chlorobenzenesulfonic acid or SO₃ with monochlorobenzene, wherein the content in the monochlorobenzene used of hydrocarbons having from 5 to 8 carbon atoms is at most 100 ppm, based on the total weight of the monochlorobenzene used, including the secondary components, to 4,4′-dichlorodiphenyl sulfone.
